(19) United States
(12) Patent Application Publication
(10) Pub. No.: US 2026/0294905 A1
(43) Pub. Date: Oct. 1, 2026

(54) USE OF PLURALITY OF COMPOUNDS IN PREPARATION OF DRUG FOR TREATING MYELOPROLIFERATIVE NEOPLASMS

(71) Applicant: THE SECOND XIANGYA HOSPITAL OF CENTRAL SOUTH UNIVERSITY, Changsha (CN)

(72) Inventors: Tuo DENG, Changsha (CN); Wanyu HU, Changsha (CN); Aini WUFUER, Changsha (CN)

(21) Appl. No.: 18/996,420

(22) PCT Filed: Aug. 21, 2023
(86) PCT No.: PCT/CN2023/113994

§ 371 Date: Jan. 17, 2025

(30) Foreign Application Priority Data
Jul. 19, 2022 (CN) 202210846177.8

(51) Int. Cl. A61K 31/506; A61K 31/4439; A61K 31/517; A61K 31/519; A61P 35/00

(52) U.S. Cl. CPC A61K 31/506; A61K 31/4439; A61K 31/517; A61K 31/519; A61P 35/00

(57) ABSTRACT

Use of five compounds in preparation of a drug for treating myeloproliferative neoplasms is provided. Said myeloproliferative neoplasms comprise polycythemia vera, essential thrombocythemia, and myelofibrosis, and especially Ruxolitinib-resistant myeloproliferative neoplasms. For patients with myeloproliferative neoplasms that are resistant to Ruxolitinib, these compounds can provide patients with continued oral drug treatment and avoid bone marrow transplantation.

### CROSS-REFERENCE TO RELATED APPLICATION

The present application claims priority to the Chinese patent application No. 202210846177.8 filed to the China National Intellectual Property Administration on Jul. 19, 2022 and entitled “USE OF PLURALITY OF COMPOUNDS IN PREPARATION OF DRUG FOR TREATING MYELOPROLIFERATIVE NEOPLASMS”, the entire content of which is incorporated into the present application by reference.

### TECHNICAL FIELD

The present disclosure relates to the technical field of medicine, and in particular to use of a plurality of compounds in preparation of a drug for treating myeloproliferative neoplasms.

### BACKGROUND

Myeloproliferative neoplasms (MPNs) refer to a group of neoplastic diseases caused by the clonal proliferation of one or more lines of relatively mature bone marrow cells. Their clinical manifestations include hyperplasia of one or more blood cells, accompanied by enlargement of the liver, spleen or lymph nodes. In 2016, the World Health Organization (WHO) conducted taxonomic revision on bone marrow tumors, and classified polycythemia vera (PV), primary myelofibrosis (PMF) and essential thrombocythemia (ET) into the scope of Philadelphia-negative classical myeloproliferative neoplasms. The MPNs are clonal hematopoietic stem cell diseases, and the main disease-driving gene mutations include JAK2V617F, CALR, and MPL mutations, where the JAK2V617F mutation is the most common type, which can be seen in 95% of PV patients, 50-60% of ET patients, and 55-65% of PMF patients. Gene activation leads to the activation of a JAK-STAT pathway, thus causing the occurrence of the disease. There are about 200,000 new patients with MPNs in the world every year, which brings a heavy burden to the medical and health system.

Before the advent of Ruxolitinib (RUX), commonly used drugs for treating the MPNs include hydroxyurea and polyethylene glycol-recombinant interferon-α2a. Hydroxyurea can only relieve symptoms but cannot inhibit clonal hematopoiesis. Long-term use of it may increase the risks of myelodysplastic syndrome and acute myelogenous leukemia. However, the use of interferon is limited by its high toxic and side effects. Ruxolitinibm, which is a JAK1/JAK2 inhibitor, is approved by FDA as a first-line drug for intermediate- and high-risk myelofibrosis (MF), and as a second-line drug for PV patients who are resistant to or intolerant of hydroxyurea (HU). Phase II and III clinical trials have suggested that RUX can reduce the spleen volume and alleviate symptoms in patients with intermediate- and high-risk MF and PV compared with an optimal therapy. However, there are also many problems in the use of Ruxolitinib. The results of the COMFORT and RESPONSE clinical trials show that MF patients who receive treatment with Ruxolitinib have more severe anemia. What is more serious is that long-term use of type I JAK inhibitors such as RUX can induce occurrence of drug resistance. Among MF patients who have received treatment for 1 year, more than 40% of patients develop drug resistance. Cross-resistance among several JAK inhibitors has also been found in clinical studies. In August 2019, the US FDA approved the novel oral JAK2 selective inhibitor Fedratinib for the treatment of intermediate- and high-risk primary or secondary (post-PV or post-ET) MF in adults, including patients who had previously received Ruxolitinib. The FDA also issued a black box warning that Fedratinib may cause encephalopathy, including the risk of Wernicke's encephalopathy. To further evaluate the effectiveness and safety of Fedratinib, a new multicenter Phase IIIb clinical trial (NCT03755518) is underway. Current JAK inhibitors are unable to significantly reduce mutant allele burden and thus have limited therapeutic potential. Bone marrow transplantation is the only method for curing MPNs, but there are still some problems to be solved. The choice of transplantation manner and regimen is still uncertain, and it is unclear whether to choose allogeneic transplantation or haploid homeotransplantation. Furthermore, the transplant-related mortality and long-term nature of MPNs must be considered during choosing of transplantation. Currently, bone marrow transplantation is mainly used for treating patients with high-risk myelofibrosis, but the timing of bone marrow transplantation for patients with other types of MPNs needs further discussion and confirmation by research. Bone marrow transplantation is expensive, and it is impossible for most patients to choose this treatment means under the current medical environment.

Although Ruxolitinib is a milestone drug for the treatment of MPNs, its current scope of application is narrow, and bone marrow suppression as a common side effect limits its application in its main indication, MF. Ruxolitinib cannot reduce the load of mutant genes, which means that treatment with Ruxolitinib cannot achieve remission of the disease at a molecular level and cannot fundamentally treat MPNs. Especially after the emergence of resistance to Ruxolitinib, limited therapeutic drugs are a major challenge currently faced.

### SUMMARY

In view of this, an objective of the present disclosure is to provide an effective, safe and reliable drug for patients with myeloproliferative neoplasms, especially those with Ruxolitinib resistance, aiming at the problems existed in the current treatment of myeloproliferative neoplasms.

In order to achieve the aforementioned objective of the present disclosure, the present disclosure provides the following technical solutions.

The present disclosure provides use of three compounds in preparation of a drug for treating myeloproliferative neoplasms. The compounds involved in the present application include: one or more of RO5126766, Agerafenib, or RAF265.

Preferably, the myeloproliferative neoplasms include polycythemia vera, essential thrombocythemia or myelofibrosis, where the myelofibrosis includes primary myelofibrosis, myelofibrosis secondary to polycythemia vera or myelofibrosis secondary to essential thrombocythemia.

The present disclosure further provides use of the five compounds in preparation of a drug for treating drug-resistant myeloproliferative neoplasms. The compounds involved in the present application include: one or more of MLN2480, RO5126766, Agerafenib, RAF265 or Belvarafenib.

In the embodiments provided by the present disclosure, the drug-resistant myeloproliferative neoplasms include myeloproliferative neoplasms with resistance to Ruxolitinib.

Preferably, the drug-resistant myeloproliferative neoplasms include drug-resistant polycythemia vera, drug-resistant essential thrombocythemia or drug-resistant myelofibrosis, where the myelofibrosis includes primary myelofibrosis, myelofibrosis secondary to polycythemia vera or myelofibrosis secondary to essential thrombocythemia.

The present disclosure further provides use of a compound in preparation of a drug for inhibiting proliferation of HEL cells. The compounds involved in the present application include: one or more of RO5126766, Agerafenib, or RAF265.

The present disclosure further provides use of five compounds in preparation of a drug for inhibiting proliferation of drug-resistant HEL cells. The compounds involved in the present application include but are not limited to: one or more of MLN2480, RO5126766, Agerafenib, RAF265 or Belvarafenib.

The present disclosure further provides use of a compound in preparation of a drug for promoting apoptosis of HEL cells. The compound involved in the present application is: RAF265.

The present disclosure further provides use of a compound in preparation of a drug for promoting apoptosis of drug-resistant HEL cells. The compound involved in the present application includes: MLN2480 and/or RAF265.

Preferably, the drug further includes a pharmaceutically-acceptable auxiliary material.

Preferably, a dosage form of the drug is an oral formulation or an injectable formulation.

The present disclosure further provides a method for treating myeloproliferative neoplasms by administering any one or more of the following:
(I). a compound;
(II). the compound and a pharmaceutically-acceptable auxiliary material; or
(III). a drug combination consisting of the compound and any other active ingredient;

The compound includes but is not limited to: one or more of RO5126766, Agerafenib or RAF265.

In some specific embodiments of the present disclosure, the myeloproliferative neoplasms include polycythemia vera, essential thrombocythemia or myelofibrosis, where the myelofibrosis includes primary myelofibrosis, myelofibrosis secondary to polycythemia vera or myelofibrosis secondary to essential thrombocythemia.

The present disclosure further provides a method for treating drug-resistant myeloproliferative neoplasms by administering any one or more of the following:
(I). a compound;
(II). the compound and a pharmaceutically-acceptable auxiliary material; or
(III). a drug combination consisting of the compound and any other active ingredient;

The compound includes but is not limited to: one or more of MLN2480, RO5126766, Agerafenib, RAF265 or Belvarafenib.

In some specific embodiments of the present disclosure, the drug-resistant myeloproliferative neoplasms include myeloproliferative neoplasms with resistance to Ruxolitinib.

In some specific embodiments of the present disclosure, the drug-resistant myeloproliferative neoplasms include drug-resistant polycythemia vera, drug-resistant essential thrombocythemia or drug-resistant myelofibrosis, where the myelofibrosis includes primary myelofibrosis, myelofibrosis secondary to polycythemia vera or myelofibrosis secondary to essential thrombocythemia.

The present disclosure further provides a method for inhibiting proliferation of HEL cells, including contacting the HEL cells with any one or more of the following:
(I). a compound;
(II). the compound and a pharmaceutically-acceptable auxiliary material; or
(III). a drug combination consisting of the compound and any other active ingredient;

The compound includes but is not limited to: one or more of RO5126766, Agerafenib or RAF265.

The present disclosure further provides a method for inhibiting proliferation of drug-resistant HEL cells, including contacting the HEL cells with any one or more of the following:
(I). a compound;
(II). the compound and a pharmaceutically-acceptable auxiliary material; or
(III). a drug combination consisting of the compound and any other active ingredient;

The compound includes but is not limited to: one or more of MLN2480, RO5126766, Agerafenib, RAF265 or Belvarafenib.

The present disclosure further provides a method for promoting apoptosis of HEL cells, including contacting the HEL cells with any one or more of the following:
(I). a compound RAF265;
(II). the compound RAF265 and a pharmaceutically-acceptable auxiliary material; or
(III). a drug combination consisting of the compound RAF265 and any other active ingredient.

The present disclosure further provides a method for promoting apoptosis of drug-resistant HEL cells, including contacting the HEL cells with any one or more of the following:
(I). a compound;
(II). the compound and a pharmaceutically-acceptable auxiliary material; or
(III). a drug combination consisting of the compound and any other active ingredient;

The compound includes but is not limited to: MLN2480 and/or RAF265. MLN2480 shows good anti-tumor activity in a preclinical model including melanoma. MLN2480 can pass the blood-brain barrier well and can be used for treating nervous system tumors such as astrocytomas and brain metastasis of melanoma. MLN2480 has passed phase I clinical trials (NCT02327169; NCT01425008), and is taken orally once a week at a recommended phase II dose (RP2D) of 420 mg/m² (not exceeding 600 mg), showing good safety and low toxic and side effects. In May 2021, we began to recruit patients with low-grade gliomas and planned to conduct phase II clinical trials (NCT04775485). MLN2480 has a molecular formula of C₁₇H₁₂C₁₂F₃N₇O₂S, a molecular weight of 506.3, and a structural formula as shown in formula I.

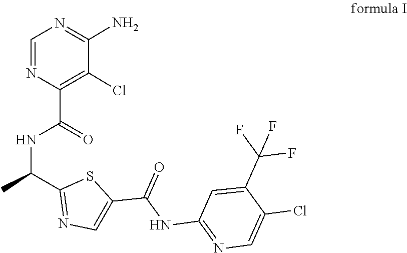 [SMILES: C[C@@H](NC(=O)c1ncnc(N)c1Cl)c1ncc(C(=O)Nc2cc(C(F)(F)F)c(Cl)cn2)s1]

RO5126766 is also known as VS-6766 or CH512766. In the first human study (NCT00773526) of CH5126766, it shows good anti-tumor activity and tolerable side effects against progressive or metastatic solid tumors. It is taken 4 consecutive days a week for 4 weeks at a recommended phase 2 dose of 2.7 mg, until occurrence of disease progression, unacceptable toxicity or patient withdrawal. CH512766 has a molecular formula of C₂₁H₁₈FN₅O₅S, a molecular weight of 471.46, and a structural formula as shown in formula II.

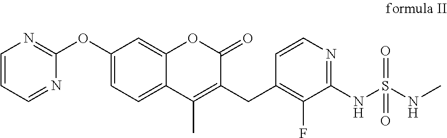 [SMILES: CNS(=O)(=O)Nc1nccc(Cc2c(C)c3ccc(Oc4ncccn4)cc3oc2=O)c1F]

Agerafenib is also known as RXDX-105 or CEP-32496. In a preclinical study of neuroblastoma, Agerafenib is able to inhibit the proliferation and colony formation of neuroblastoma cells in vitro, and had a synergistic effect with traditional chemotherapy drugs. At the same time, in a neuroblastoma mouse model, it is able to inhibit tumor growth of and prolong the survival of mice significantly. In a phase I/Ib clinical trial (NCT01877811) for patients with non-small cell lung cancer, RXDX-105 shows good anti-tumor activity, and the recommended phase 2 dose of it is 275 mg per day. Agerafenib has a molecular formula of C₂₄H₂₂F₃N₅O₅, a molecular weight of 517.46, and a structural formula as shown in formula III.

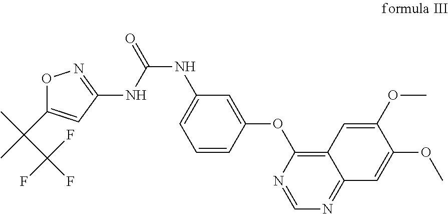 [SMILES: COc1cc2ncnc(Oc3cccc(NC(=O)Nc4cc(C(C)(C)C(F)(F)F)on4)c3)c2cc1OC]

RAF265 has a molecular formula of C₂₄H₁₆F₆N₆O, a molecular weight of 518.41, and a structural formula as shown in formula IV.

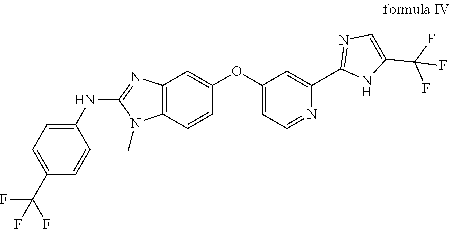 [SMILES: Cn1c(Nc2ccc(C(F)(F)F)cc2)nc2cc(Oc3ccnc(-c4ncc(C(F)(F)F)[nH]4)c3)ccc21]

Belvarafenib has a molecular formula of C₂₃H₁₆ClFN₆OS, a molecular weight of 478.93, and a structural formula as shown in formula V.

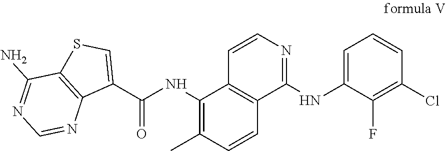 [SMILES: Cc1ccc2c(Nc3cccc(Cl)c3F)nccc2c1NC(=O)c1csc2c(N)ncnc12]

It can be seen from the aforementioned technical solutions that, the present disclosure provides the use of five compounds in preparation of a drug for treating myeloproliferative neoplasms. The myeloproliferative neoplasms include polycythemia vera, essential thrombocythemia or myelofibrosis and ruxolitinib-resistant myeloproliferative neoplasms, where the myelofibrosis includes primary myelofibrosis, myelofibrosis secondary to polycythemia vera or myelofibrosis secondary to essential thrombocythemia. The technical effects of the present disclosure are as follows.

The use of compounds RO5126766, Agerafenib, and RAF265 for the treatment of myeloproliferative neoplasms provides new treatment routes for patients with myeloproliferative neoplasms and provides more options for clinicians and patients. For patients with myeloproliferative neoplasms that are resistant to Ruxolitinib, five compounds (MLN2480, RO5126766, Agerafenib, RAF265 or Belvarafenib) can provide patients with continued oral drug treatment and avoid bone marrow transplantation. The aforementioned compounds can be chemically synthesized at a lower cost than that of biological formulations. Moreover, the compounds in the present application have all passed phase I clinical trials and have good safety. Some of them have successfully passed phase II clinical trials and will be used for clinical treatment in the future, with good prospects for clinical application.

### BRIEF DESCRIPTION OF THE DRAWINGS

FIG. 1 shows a diagram illustrating a result of establishing a model HEL^{RE} of Ruxolitinib-resistant myeloproliferative neoplasm cells in Example 1;

FIG. 2 shows a diagram illustrating a cell proliferation result of two types of myeloproliferative neoplasm cells subjected to compound treatment, as detected by a CellTiter-Lumi™ luminescence method in Example 2, where a is a panel showing a cell proliferation result of HEL treated with MLN2480; b is a panel showing a cell proliferation result of HEL treated with RO5126766; c is a panel showing a cell proliferation result of HEL treated with Agerafenib; d is a panel showing a cell proliferation result of HEL treated with RAF265; e is a panel showing a cell proliferation result of HEL treated with Belvarafenib; and see Table 1 for specific numerical values;

FIG. 3 shows a result of cell apoptosis of two types of myeloproliferative neoplasm cells subjected to compound treatment, as stained with Annexin V-PI and detected by flow cytometry in Example 3, where a is a panel showing a result of cell apoptosis of HEL treated with MLN2480; b is a panel showing a result of cell apoptosis of HEL treated with RO5126766; c is a panel showing a result of cell apoptosis of HEL treated with Agerafenib; d is a panel showing a result of cell apoptosis of HEL treated with RAF265; e is a panel showing a result of cell apoptosis of HEL treated with Belvarafenib; and see Table 2 for specific numerical values;

FIG. 4 shows a diagram illustrating a cell proliferation result of two types of Ruxolitinib-resistant myeloproliferative neoplasm cells subjected to compound treatment, as detected by a CellTiter-Lumi™ luminescence method in Example 4, where a is a panel showing a cell proliferation result of HEL^{RE} treated with MLN2480; b is a panel showing a cell proliferation result of HEL^{RE} treated with RO5126766; c is a panel showing a cell proliferation result of HEL^{RE} treated with Agerafenib; d is a panel showing a cell proliferation result of HEL^{RE} treated with RAF265; e is a panel showing a cell proliferation result of HEL^{RE} treated with Belvarafenib; and see Table 3 for specific numerical values;

FIG. 5 shows a result of cell apoptosis of two types of Ruxolitinib-resistant myeloproliferative neoplasm cells subjected to compound treatment, as stained with Annexin V-PI and detected by flow cytometry in Example 5, where a is a panel showing a result of cell apoptosis of HEL^{RE} treated with MLN2480; b is a panel showing a result of cell apoptosis of HEL^{RE} treated with RO5126766; c is a panel showing a result of cell apoptosis of HEL^{RE} treated with Agerafenib; d is a panel showing a result of cell apoptosis of HEL^{RE} treated with RAF265; e is a panel showing a result of cell apoptosis of HEL^{RE} treated with Belvarafenib; and see Table 4 for specific numerical values.

### DETAILED DESCRIPTION OF THE EMBODIMENTS

The present disclosure discloses use of five compounds in preparation of a drug for treating myeloproliferative neoplasms, the implementation of process parameters can be appropriately improved by those skilled in the art with reference to the content of this article. It should be particularly noted that all similar substitutions and modifications are obvious to those skilled in the art, and they are deemed to be included in the present disclosure. The methods and use of the present disclosure have been described through preferred embodiments. Relevant personnel can obviously modify or appropriately change and combine the methods and use described herein to implement and apply the technology of the present disclosure, without departing from the content, spirit and scope of the present disclosure.

In the present disclosure, the inhibitory effects of five compounds on myeloproliferative neoplasm cells (resistant and non-resistant) are clarified by using cell line models.

In some embodiments, in the present disclosure a Ruxolitinib-resistant cell model HEL^{RE} is established based on a commonly used human-derived myeloproliferative neoplasm cell line containing a JAK2-V617F mutation, i.e., HEL cells (human erythroleukemia cell line). A method for constructing a drug-resistant model is to start addition of Ruxolitinib at a concentration lower than IC50 of naive cells, which is slowly incremented to a high concentration, prevent the cells from being killed, and verify whether the model construction is successful by comparing the IC50s.

In some embodiments, in the present disclosure increasing concentrations of compounds are used for treating myeloproliferative neoplasm cell lines (HEL), and the proliferation of cells is detected by a CellTiter-Lumi™ luminescence method. The result shows that RO5126766, Agerafenib, and RAF265 can successfully inhibit the proliferation of the HEL cells.

In some embodiments, in the present disclosure increasing concentrations of compounds are used for treating myeloproliferative neoplasm cell lines (HEL), and cell apoptosis is detected by Annexin V-PI staining followed by flow cytometry. The result shows that RAF265 can promote the apoptosis of the HEL cells.

It can be seen that the compounds RO5126766, Agerafenib, and RAF265 can be used for treating bone marrow myeloproliferative neoplasm diseases, and RAF265 is more effective.

Further, in some embodiments, in the present disclosure increasing concentrations of compounds are used for treating Ruxolitinib-resistant myeloproliferative neoplasm cells (HEL^{RE}), and cell proliferation is detected by a CellTiter-Lumi™ luminescence method. The result shows that these compounds can successfully inhibit the proliferation of HEL^{RE} cells.

In some embodiments, in the present disclosure increasing concentrations of compounds are used for treating Ruxolitinib-resistant myeloproliferative neoplasm cells (HEL^{RE}), and cell apoptosis is detected by Annexin V-PI staining followed by flow cytometry. The result shows that MLN2480 and RAF265 can promote the apoptosis of the HEL^{RE} cells.

It can be seen that the five compounds can be used for treating Ruxolitinib-resistant myeloproliferative neoplasms, and MLN2480 and RAF265 are more effective.

Further, the present disclosure provides use of the compounds in preparation of a drug for inhibiting the proliferation of HEL cells and promoting the apoptosis of HEL cells.

In view of the above, the present disclosure provides use of three compounds in preparation of a drug for treating myeloproliferative neoplasms; the compounds include but are not limited to: one or more of RO5126766, Agerafenib or RAF265.

Further, the myeloproliferative neoplasms are polycythemia vera, essential thrombocythemia and myelofibrosis (including primary myelofibrosis, myelofibrosis secondary to polycythemia vera and myelofibrosis secondary to essential thrombocythemia) and drug-resistant myeloproliferative neoplasms.

In some embodiments, the drug-resistant myeloproliferative neoplasms are Ruxolitinib-resistant myeloproliferative neoplasms.

In some embodiments, the drug-resistant myeloproliferative neoplasms are drug-resistant polycythemia vera, drug-resistant myelofibrosis (including primary myelofibrosis, myelofibrosis secondary to polycythemia vera, and myelofibrosis secondary to essential thrombocythemia), and drug-resistant essential thrombocythemia.

The drug is MLN2480, RO5126766, Agerafenib, RAF265, and Belvarafenib.

Further, the drug also includes a pharmaceutically-acceptable auxiliary material.

The drug can be in any dosage form in the current pharmaceutical field, including oral formulations or injection formulations.

Each drug dosage form can be prepared with an appropriate acceptable accessory material selected according to the actual needs of the dosage form, which belongs to conventional dosage form preparation technology in the art. The drug is prepared into dosage forms such as capsules, tablets, injection powders, and the like.

The reagents and instruments used in the present disclosure all can be purchased from the market.

The present disclosure will be further described hereafter in conjunction with examples:

### Example 1. Establishment of a Common Ruxolitinib-Resistant Cell Model (HEL^{RE})

### I. Materials and Methods

### 1. Cell Lines

HELs (Human erythroleukemia cell lines) and Ruxolitinib-resistant HEL cells were both cultured in a RPMI medium (Gibco) containing 20% heat-inactivated fetal bovine serum (Gibco) and 1% penicillin/streptomycin.

A Ruxolitinib-resistant HEL model, i.e., a HEL^{RE} model, was constructed by starting the addition of Ruxolitinib at a concentration lower than the IC50 of naïve cells and slowly incrementing the concentration to a high concentration, and preventing the cells from being killed. Our starting concentration was 0.1 μM. The drug was added upon occurrence of cell proliferation. The drug addition gradient was an increment at a multiple of 1.25, and the final concentration was 2.0 μM. Stable drug-resistant cells were obtained after 4-6 weeks.

### 2. Compounds

Ruxolitinib and other compounds were all purchased from Selleck, dissolved in DMSO with a stock solution concentration of 10 mM, and cryopreserved at −80° C. The working solution was diluted to a specified multiple using a RPMI medium and then used for treating cells. Ruxolitinib was specifically ruxolitinib phosphate.

### 3. In Vitro Inhibition Test

To detect the antiproliferative effect of the inhibitor, the aforementioned cell lines were cultured at 3,000 cells/100 μL of the system per well, added with increasing concentrations of Ruxolitinib (concentration gradients of HEL cells: 0, 0.1, 1, 2.5, 5 μM; or 0, 0.1, 0.3, 1, 3, 10 μM), and added with DMSO to make up to the same amount. 4 parallel replicate groups were set up, and 3 blank wells (wells containing culture media without cells) were set up. After 48 hours, the cell proliferation was detected by a CellTiter-Lumi™ luminescence method (Beyotime). data was read from a multifunctional microplate reader and the IC50 was calculated from the data through GraphPadprism.

A calculation formula of the cell proliferation rate was: cell proliferation rate=(Luminescence value of the group added with the drug-average Luminescence value of the blank wells)/(Luminescence value of the DMSO control group-average Luminescence value of the blank wells)×100%.

A method for evaluating whether the model was successfully constructed was to compare the IC50s of resistant cells and naïve cells. A ratio of them was a drug resistance index, and the ratio being greater than 3 indicated that the construction was successful.

### II. Results Analysis

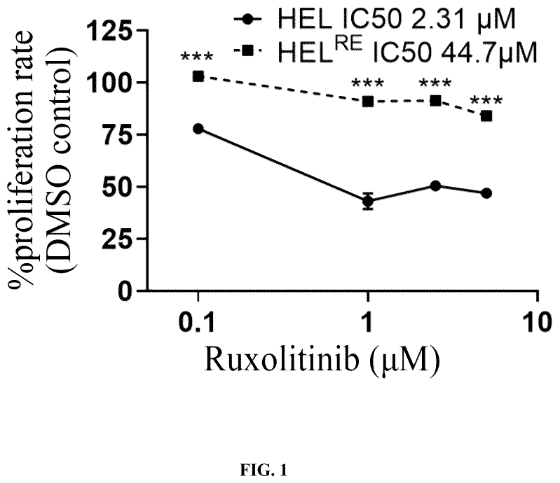

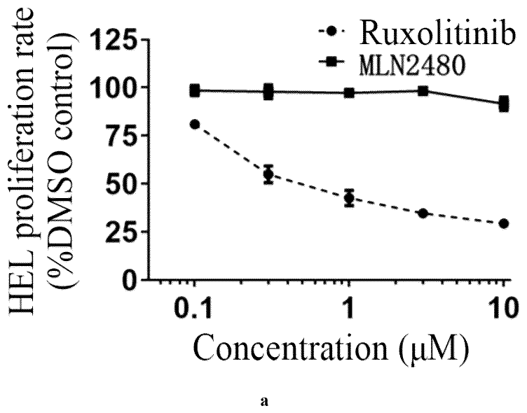

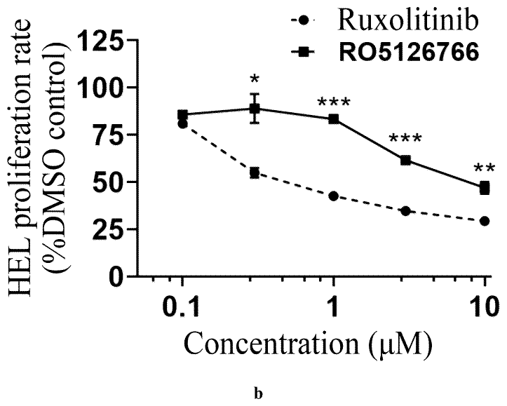

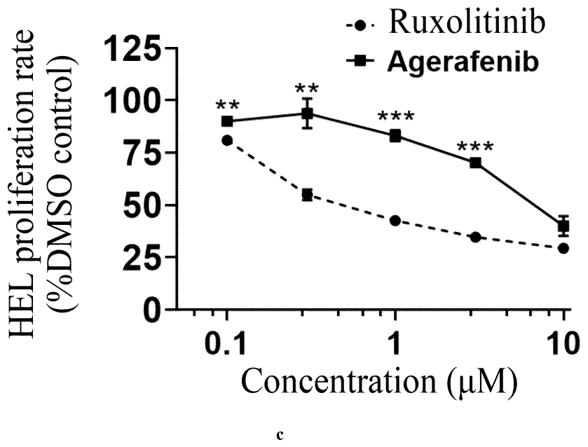

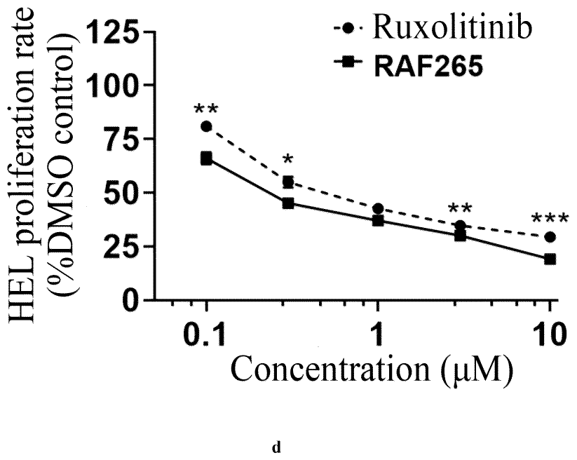

In FIG. 1, the IC50 of the HEL cells was 2.31 (1.42-4.36) μM, and the IC50 of the HEL^{RE} cells was 44.7 (16.0-370) μM, so that the drug resistance index was 19.0, indicating successful construction of the drug-resistant model HEL^{RE}. The proliferation rate result in the figure was shown as mean±standard deviation. The comparison of the proliferation rates of the HEL^{RE} cells vs the HEL cells in the figure was performed by employing a t-test (***p<0.001), and the IC50 was shown as an average. Results were presented as means (with a confidence interval of 95%) in the analysis.

### Example 2: Some Compounds can Inhibit the Proliferation of Myeloproliferative Neoplasm Cells

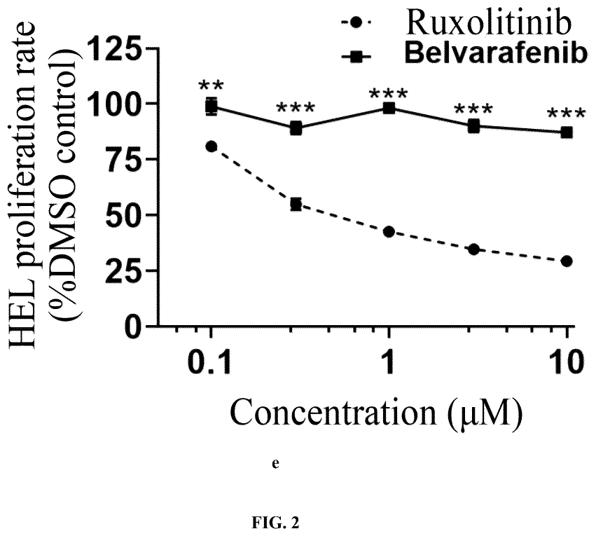

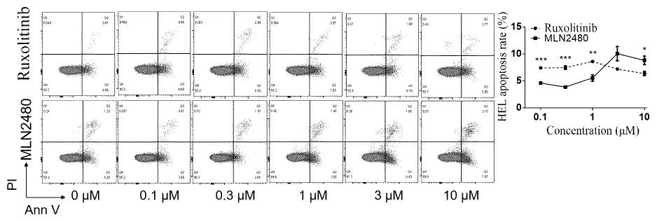

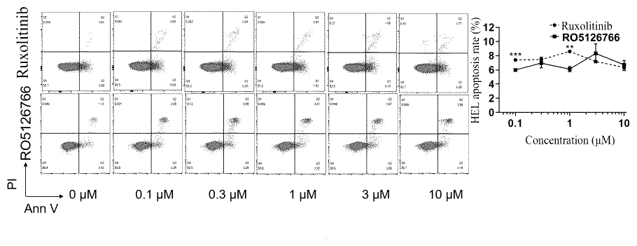

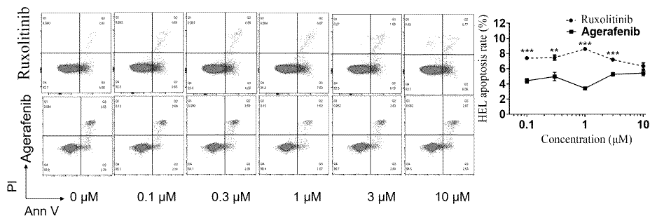

In order to detect the effects of the compounds on the proliferation abilities of drug-resistant myeloproliferative neoplasm cells, the method was to treat the HEL naïve cell lines with increasing concentrations of Ruxolitinib and the compounds respectively, and to detect the proliferation of the cells by a CellTiter-Lumi™ luminescence method. The method was the same as that of Example 1, the results were shown in FIG. 2, and the numerical values were shown in Table 1.

TABLE 1
% average proliferation rate ± standard
deviation of HEL cells treated with drugs

| Drugs | 0 μM | 0.1 μM | 0.3 μM | 1 μM | 3 μM | 10 μM |
|---|---|---|---|---|---|---|
| Ruxolitinib | 100 ± | 80.9 ± | 54.9 ± | 42.7 ± | 34.7 ± | 29.4 ± |
| (FIG. 2) | 1.47 | 1.35 | 4.35 | 4.05 | 1.14 | 0.49 |
| MLN2480 | 100 ± | 98.5 ± | 97.9 ± | 97.3 ± | 98.3 ± | 97.6 ± |
| (FIG. 2a) | 1.35 | 2.62 | 3.55 | 0.78 | 0.65 | 3.47 |
| RO5126766 | 100 ± | 85.8 ± | 89.0 ± | 83.4 ± | 61.6 ± | 47.1 ± |
| (FIG. 2b) | 6.42 | 2.75 | 13.5 | 3.31 | 3.79 | 5.47 |
| Agerafenib | 100 ± | 90.1 ± | 93.8 ± | 83.2 ± | 70.3 ± | 40.1 ± |
| (FIG. 2c) | 5.18 | 3.01 | 12.4 | 4.45 | 3.47 | 8.26 |
| RAF265 | 100 ± | 66.1 ± | 45.2 ± | 37.0 ± | 30.0 ± | 19.2 ± |
| (FIG. 2d) | 7.75 | 4.71 | 2.96 | 2.18 | 0.130 | 0.990 |
| Belvarafenib | 100 ± | 98.9 ± | 89.2 ± | 98.2 ± | 90.1 ± | 87.3 ± |
| (FIG. 2e) | 1.46 | 6.41 | 4.68 | 3.19 | 4.72 | 2.39 |

FIG. 2a reflected that in the HEL cells, the drug concentrations (% average proliferation rate±standard deviation) of the groups treated with Ruxolitinib were: 0 μM (100±1.47) (not shown), 0.1 μM (80.9±1.35), 0.3 μM (54.9±4.35), 1 μM (42.7±4.05), 3 μM (34.7±1.14), and 10 μM (29.4±0.49); the drug concentrations (average proliferation rates) of the groups treated with MLN2480 were: 0μ M (100±1.35) (not shown), 0.1 μM (98.5±2.62), 0.3μ M (97.9±3.55), 1 μM (97.3±0.78), 3 μM (98.3±0.65), and 10 μM (97.6±3.47). This suggested that MLN2480 could not inhibit the proliferation of the HEL cells. FIG. 2b reflected that in the HEL cells, the drug concentrations (average proliferation rates) of the groups treated with RO5126766 were: 0 μM (100±6.42) (not shown), 0.1 μM (85.8±2.75), 0.3 μM (89.0±13.5), 1 μM (83.4±3.31), 3 μM (61.6±3.79), and 10 μM (47.1±5.47). This suggested that RO5126766 could inhibit the proliferation of the HEL cells, and this effect increased with increase in the drug concentration. FIG. 2c reflected that in the HEL cells, the drug concentrations (average proliferation rates) of the groups treated with Agerafenib were: 0 μM (100±5.18) (not shown), 0.1 μM (90.1±3.01), 0.3 μM (93.8±12.4), 1 μM (83.2±4.45), 3 μM (70.3±3.47), and 10 μM (40.1±8.26). This suggested that Agerafenib could inhibit the proliferation of the HEL cells, and this effect was increased with the increase of the drug concentration. FIG. 2d reflected that in the HEL cells, the drug concentrations (average proliferation rates) of the groups treated with RAF265 were: 0 μM (100±7.75) (not shown), 0.1 μM (66.1±4.71), 0.3 μM (45.2±2.96), 1 μM (37.0±2.18), 3 μM (30.0±0.130), and 10 μM (19.2±0.990). This suggested that RAF265 could inhibit the proliferation of the HEL cells, this effect was increased with the increase of the drug concentration, and its inhibitory effect was better than that of Ruxolitinib. FIG. 2e reflected that in the HEL cells, the drug concentrations (average proliferation rates) of the groups treated with Belvarafenib were: 0 μM (100±1.46) (not shown), 0.1 μM (98.9±6.41), 0.3 μM (89.2±4.68), 1 μM (98.2±3.19), 3 μM (90.1±4.72), and 10 μM (87.3±2.39). This suggested that Belvarafenib could not inhibit the proliferation of the HEL cells. The comparison in proliferation rates of the groups treated with various concentrations of compounds and the group treated with DMSO was conducted by employing a t-test (*p<0.05, **p<0.01, ***p<0.001).

### Example 3: Some Compounds can Promote Apoptosis of Myeloproliferative Neoplasm Cells

### I. Materials and Methods

1. the cell lines and inhibitors were the same as those in Example 1.
2. detection of cell apoptosis

To detect the apoptosis-promoting effect of the inhibitors, HEL naïve cell strains were treated with Ruxolitinib and the compounds for 24 hours respectively (with concentrations of: 0, 0.1, 0.5, 1 μM or 0, 0.1, 0.3, 1, 3, 10 μM), and added with DMSO to make up to the same amount. 3 parallel replicate groups were set up, and cell apoptosis was detected by Annexin V and PI staining followed by flow cytometry.

A calculation formula of cell apoptosis rate was: cell apoptosis rate=the ratio of early apoptotic cells (Annexin V⁺/PI⁻)+the ratio of late apoptotic cells and necrotic cells (Annexin V⁺/PI⁺).

### II. Results Analysis

The numerical values were shown in Table 2.

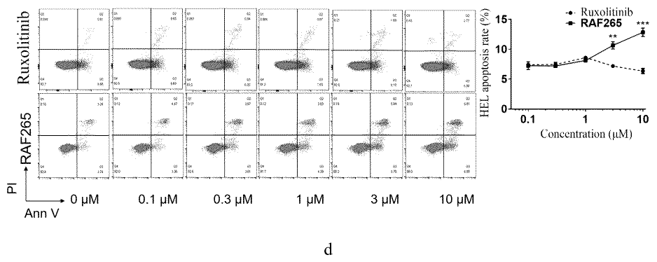

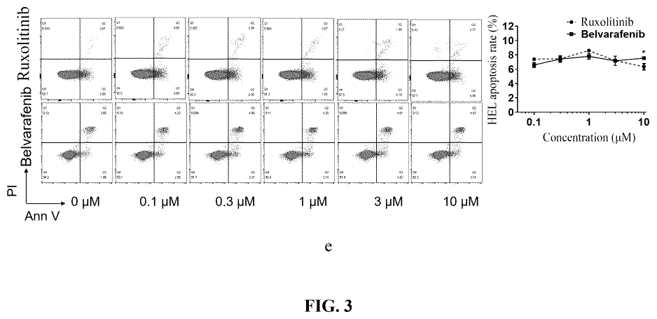

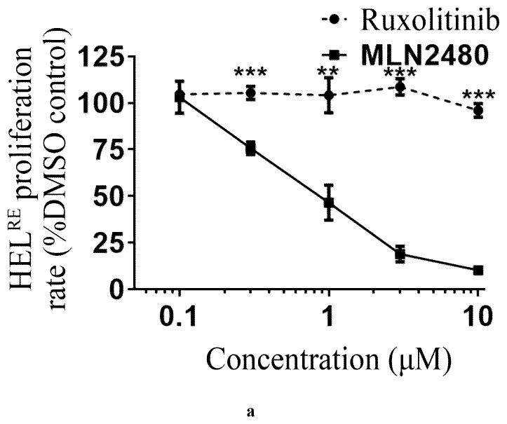

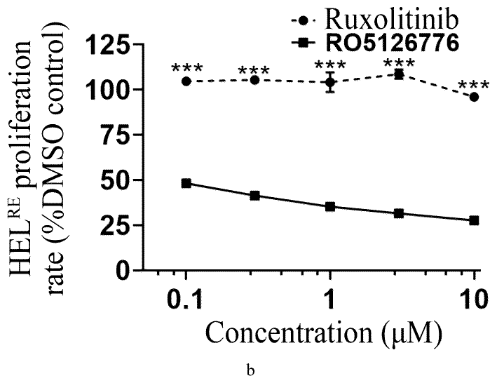

TABLE 2
% average apoptosis rate ± standard
deviation of HEL cells treated with drugs

| Drugs | 0 μM | 0.1 μM | 0.3 μM | 1 μM | 3 μM | 10 μM |
|---|---|---|---|---|---|---|
| Ruxolitinib | 7.39 ± | 7.40 ± | 7.46 ± | 8.61 ± | 7.19 ± | 6.36 ± |
| (FIG. 3) | 0.387 | 0.059 | 0.679 | 0.403 | 0.209 | 0.036 |
| MLN2480 | 6.09 ± | 4.61 ± | 3.85 ± | 5.53 ± | 10.04 ± | 8.8 ± |
| (FIG. 3a) | 0.756 | 2.768 | 0.101 | 1.22 | 2.663 | 1.063 |
| RO5126766 | 4.34 ± | 6.00 ± | 6.96 ± | 6.11 ± | 8.32 ± | 6.73 ± |
| (FIG. 3b) | 1.39 | 0.290 | 1.16 | 0.640 | 2.69 | 1.20 |
| Agerafenib | 6.30 ± | 4.41 ± | 4.98 ± | 3.42 ± | 5.27 ± | 5.43 ± |
| (FIG. 3c) | 0.450 | 0.630 | 1.09 | 0.140 | 0.490 | 0.720 |
| RAF265 | 5.23 ± | 7.24 ± | 7.19 ± | 8.11 ± | 10.7 ± | 12.8 ± |
| (FIG. 3d) | 1.05 | 1.30 | 0.400 | 0.260 | 1.22 | 1.44 |
| Belvarafenib | 5.56 ± | 6.58 ± | 7.42 ± | 7.80 ± | 7.17 ± | 7.55 ± |
| (FIG. 3e) | 0.130 | 0.760 | 0.950 | 0.700 | 1.31 | 0.160 |

In FIG. 3a, the drug concentrations (% average apoptosis rate±standard deviation) of the groups of HEL cells treated with Ruxolitinib were: 0 μM (7.39±0.387) (not shown), 0.1 μM (7.40±0.059), 0.3 μM (7.46±0.679), 1 μM (8.61±0.403), 3 μM (7.19±0.209), and 10 μM (6.36±0.036); and the drug concentrations (apoptosis rates) of the groups treated with MLN2480 were 0 μM (6.09±0.756) (not shown), 0.1 μM (4.61±2.768), 0.3 μM (3.85±0.101), 1 μM (5.53±1.22), 3 μM (10.04±2.663), and 10 μM (8.8±1.063); and this suggested that MLN2480 could not promote the apoptosis of myeloproliferative neoplasm cells. FIG. 3b reflected that in the HEL cells, the drug concentrations (average apoptosis rates) of the groups treated with RO5126766 were: 0 μM (4.34±1.39) (not shown), 0.1 μM (6.00±0.290), 0.3 M (6.96±1.16), 1 μM (6.11±0.640), 3 μM (8.32±2.69), and 10 μM (6.73±1.20). This suggested that RO5126766 could not promote apoptosis of the HEL cells. FIG. 3c reflected that in the HEL cells, the drug concentrations (average apoptosis rates) of the groups treated with Agerafenib were: 0 μM (6.30±0.450) (not shown), 0.1 μM (4.41±0.630), 0.3 μM (4.98±1.09), 1 μM (3.42±0.140), 3 μM (5.27=0.490), and 10 μM (5.43±0.720). This suggested that Agerafenib could not promote apoptosis of the HEL cells. FIG. 3d reflected that in the HEL cells, the drug concentrations (average apoptosis rates) of the groups treated with RAF265 were: 0 μM (5.23±1.05) (not shown), 0.1 μM (7.24±1.30), 0.3 μM (7.19±0.400), 1 μM (8.11±0.260), 3 μM (10.7±1.22), and 10 μM (12.8±1.44). This suggested that RAF265 could promote the apoptosis of the HEL cells, this effect was increased with the increase of the drug concentration, and its inhibitory effect was better than that of Ruxolitinib. FIG. 3e reflected that in the HEL cells, the drug concentrations (average apoptosis rates) of the groups treated with Belvarafenib were: 0 μM (5.56±0.130) (not shown), 0.1 μM (6.58±0.760), 0.3 μM (7.42±0.950), 1 μM (7.80±0.700), 3 μM (7.17±1.31), and 10 μM (7.55±0.160). This suggested that Belvarafenib could not promote apoptosis of the HEL cells. The comparison in the apoptosis rates of the groups treated with various concentrations of compounds and the group treated with DMSO was conducted by employing a t-test (*p<0.05, **p<0.01, ***p<0.001).

### Example 4: Five Compounds can Inhibit the Proliferation of Drug-Resistant Myeloproliferative Neoplasm Cells

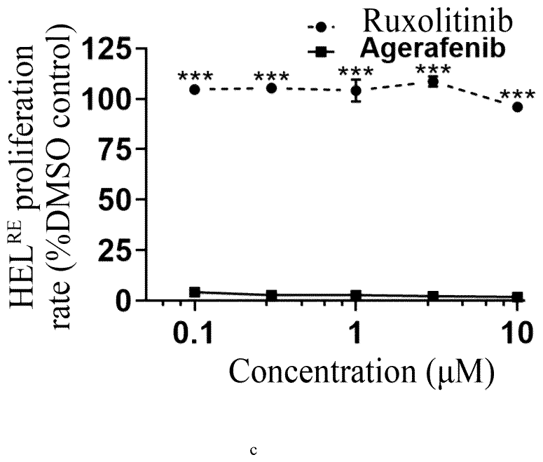

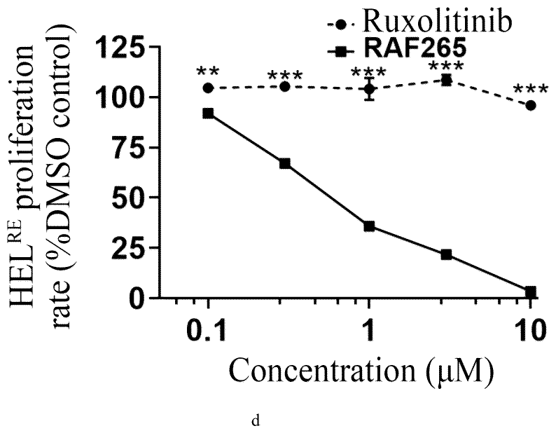

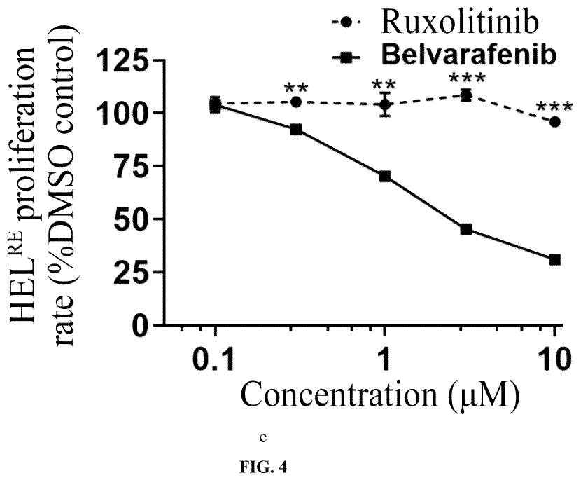

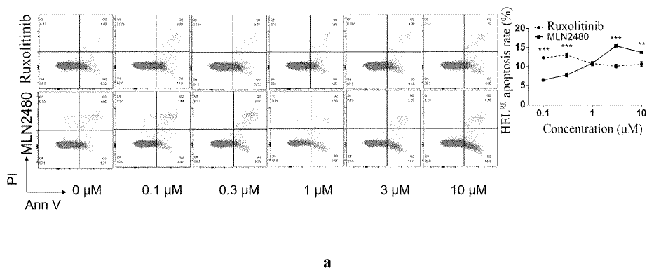

In order to detect the effects of five compounds on the proliferation abilities of drug-resistant myeloproliferative neoplasm cells, the method was to treat HEL^{RE} with increasing concentrations of Ruxolitinib and the five compounds respectively, and to detect the proliferation of the cells by a CellTiter-Lumi™ luminescence method. The method was the same as that of Example 1, the results were shown in FIG. 4, and the numerical values were shown in Table 3.

TABLE 3
% average proliferation rate ± standard
deviation of HEL^{RE} cells treated with drugs

| Drugs | 0 μM | 0.1 μM | 0.3 μM | 1 μM | 3 μM | 10 μM |
|---|---|---|---|---|---|---|
| Ruxolitinib | 100 ± | 105 ± | 105 ± | 104 ± | 109 ± | 96.0 ± |
| (FIG. 4) | 3.79 | 2.08 | 3.59 | 9.46 | 4.39 | 3.69 |
| MLN2480 | 100 ± | 103.1 ± | 75.1 ± | 46.5 ± | 18.9 ± | 10.3 ± |
| (FIG. 4a) | 4.37 | 8.58 | 3.54 | 9.45 | 4.22 | 0.490 |
| RO5126766 | 100 ± | 48.1 ± | 41.4 ± | 35.2 ± | 31.5 ± | 27.7 ± |
| (FIG. 4b) | 1.82 | 3.37 | 3.16 | 2.89 | 1.90 | 2.34 |
| Agerafenib | 100 ± | 4.26 ± | 2.77 ± | 2.75 ± | 2.21 ± | 1.86 ± |
| (FIG. 4c) | 4.00 | 0.460 | 0.430 | 0.940 | 0.0400 | 0.230 |
| RAF265 | 100 ± | 92.0 ± | 67.2 ± | 35.8 ± | 21.8± | 3.45 ± |
| (FIG. 4d) | 4.58 | 3.92 | 3.70 | 3.88 | 1.48 | 0.580 |
| Belvarafenib | 100 ± | 104 ± | 92.4 ± | 70.4 ± | 45.4 ± | 31.1 ± |
| (FIG. 4e) | 6.75 | 6.13 | 2.45 | 3.11 | 2.33 | 3.60 |

FIG. 4a reflected that in the HEL^{RE} cells, the drug concentrations (% average proliferation rate±standard deviation) of the groups treated with Ruxolitinib were: 0 μM (100±3.79) (not shown), 0.1μ M (105±2.08), 0.3 μM (105±3.59), 1 μM (104±9.46), 3 μM (109±4.39), and 10 μM (96.0±3.69); and the drug concentrations (average proliferation rates) of the groups treated with MLN2480 were: 0 μM (100±4.37) (not shown), 0.1 μM (103.1±8.58), 0.3 μM (75.1±3.54), 1 μM (46.5±9.45), 3 μM (18.9±4.22), and 10 μM (10.3±0.490). This suggested that MLN2480 could inhibit the proliferation of drug-resistant myeloproliferative neoplasm cells, and this effect was increased with the increase of the drug concentration. FIG. 4b reflected that in the HEL^{RE} cells, the drug concentrations (average proliferation rates) of the groups treated with RO5126766 were: 0 μM (100±1.82) (not shown), 0.1 μM (48.1±3.37), 0.3 μM (41.4±3.16), 1 μM (35.2±2.89), 3 μM (31.5±1.90), and 10 μM (27.7±2.34). This suggested that RO5126766 could inhibit the proliferation of the HEL^{RE} cells, and this effect increased with increase in the drug concentration. FIG. 4c reflected that in the HEL^{RE} cells, the drug concentrations (average proliferation rates) of the groups treated with Agerafenib were: 0 μM (100±4.00) (not shown), 0.1 μM (4.26±0.460), 0.3 μM (2.77±0.430), 1 μM (2.75±0.940), 3 μM (2.21±0.0400), and 10 μM (1.86±0.230). This suggested that Agerafenib could inhibit the proliferation of the HEL^{RE} cells, and this effect was increased with the increase of the drug concentration. FIG. 4d reflected that in the HEL^{RE} cells, the drug concentrations (average proliferation rates) of the groups treated with RAF265 were: 0 μM (100±4.58) (not shown), 0.1 μM (92.0±3.92), 0.3 μM (67.2±3.70), 1 μM (35.8±3.88), 3 μM (21.8±1.48), and 10 μM (3.45±0.580). This suggested that RAF265 could inhibit the proliferation of the HEL^{RE} cells, and this effect increased with increase in the drug concentration. FIG. 4e reflected that in the HEL^{RE} cells, the drug concentrations (average proliferation rates) of the groups treated with Belvarafenib were: 0 μM (100±6.75) (not shown), 0.1 μM (104±6.13), 0.3 μM (92.4±2.45), 1 μM (70.4=3.11), 3 μM (45.4±2.33), and 10 μM (31.1±3.60). This suggested that Belvarafenib could inhibit the proliferation of the HEL^{RE} cells, and this effect increased with increase in the drug concentration. The comparison in proliferation rates of the groups treated with various concentrations of compounds and the group treated with corresponding concentrations of Ruxolitinib was conducted by employing a t-test (*p<0.05, **p<0.01, ***p<0.001).

### Example 5: Some Compounds can Promote Apoptosis of Drug-Resistant Myeloproliferative Neoplasm Cells

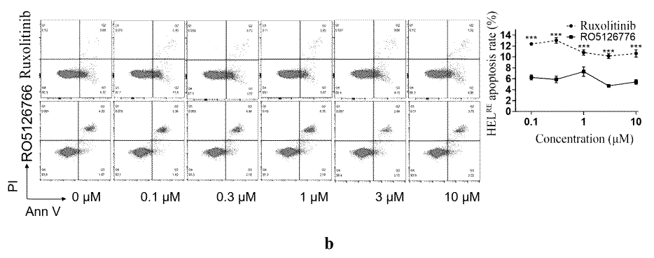

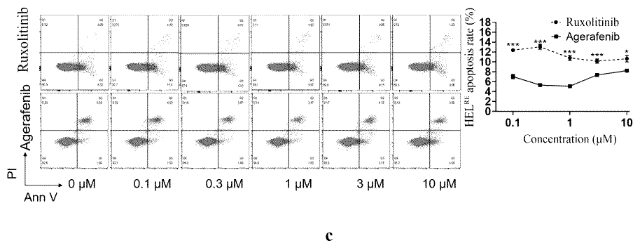

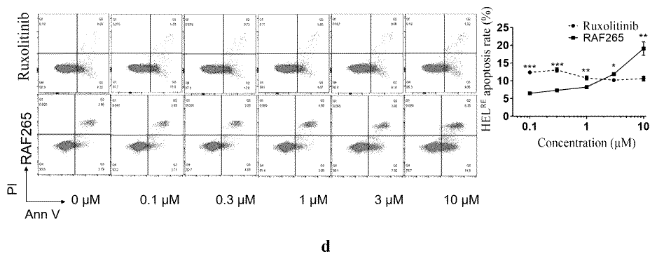

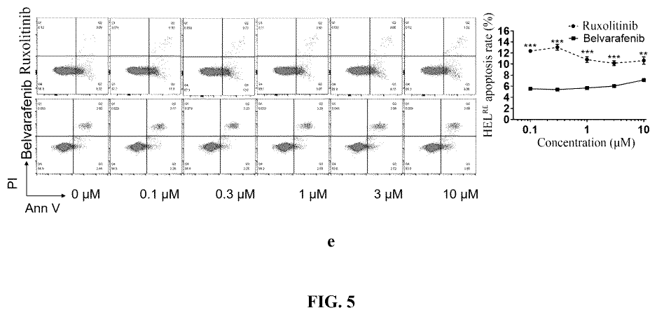

In order to detect the effects of compounds on the abilities of drug-resistant myeloproliferative neoplasm cells, the method was to treat HEL^{RE} with increasing concentrations of Ruxolitinib and the compounds, and to detect cell apoptosis by Annexin V and PI staining followed by flow cytometry. The method was the same as that of Example 3, the results were shown in FIG. 5, and the numerical values were shown in Table 4.

TABLE 4
% average apoptosis rate ± standard
deviation of HEL-RE cells treated with drugs

| Drugs | 0 μM | 0.1 μM | 0.3 μM | 1 μM | 3 μM | 10 μM |
|---|---|---|---|---|---|---|
| Ruxolitinib | 8.89 ± | 12.4 ± | 13.0 ± | 10.8 ± | 10.2 ± | 10.6 ± |
| (FIG. 5) | 1.190 | 0.540 | 1.03 | 1.02 | 0.880 | 1.33 |
| MLN2480 | 6.69 ± | 6.54 ± | 7.80 ± | 10.9 ± | 15.5 ± | 13.8 ± |
| (FIG. 5a) | 1.140 | 0.560 | 0.900 | 0.390 | 0.580 | 0.775 |
| RO5126766 | 6.12 ± | 6.25 ± | 5.88 ± | 7.32 ± | 4.71 ± | 5.42 ± |
| (FIG. 5b) | 0.270 | 0.920 | 1.25 | 1.74 | 0.350 | 0.830 |
| Agerafenib | 7.60 ± | 7.05 ± | 5.33 ± | 5.11 ± | 7.37 ± | 8.22 ± |
| (FIG. 5c) | 1.74 | 0.920 | 0.43 | 0.210 | 0.220 | 0.390 |
| RAF265 | 6.23 ± | 6.48 ± | 7.31 ± | 8.24 ± | 11.9 ± | 19.1 ± |
| (FIG. 5d) | 0.300 | 0.480 | 0.140 | 0.730 | 0.520 | 3.75 |
| Belvarafenib | 5.15 ± | 5.55 ± | 5.40 ± | 5.71 ± | 6.05 ± | 7.13 ± |
| (FIG. 5e) | 0.730 | 0.440 | 0.570 | 0.260 | 0.430 | 0.450 |

In FIG. 5a, the drug concentrations (% average apoptosis rate±standard deviation) of the groups of HEL^{RE} cells treated with Ruxolitinib were: 0 μM (8.89±1.19) (not shown), 0.1 μM (12.4±0.540), 0.3 μM (13.0±1.03), 1 μM (10.8±1.02), 3 μM (10.2±0.880), and 10 μM (10.6±1.33); and the drug concentrations (apoptosis rates) of the groups treated with MLN2480 were 0 μM (6.69±1.14) (not shown), 0.1 μM (6.54±0.560), 0.3 μM (7.80±0.900), 1 μM (10.9±0.390), 3 μM (15.5±0.580), and 10 μM (13.8±0.775). This suggested that MLN2480 could promote apoptosis of Ruxolitinib-resistant myeloproliferative neoplasm cells, and this effect was increased with the increase of the drug concentration. FIG. 5b reflected that in the HEL^{RE} cells, the drug concentrations (average apoptosis rates) of the groups treated with RO5126766 were: 0 μM (6.12±0.270) (not shown), 0.1 μM (6.25±0.920), 0.3 μM (5.88±1.25), 1 μM (7.32±1.74), 3 μM (4.71±0.350), and 10 μM (5.42±0.830). This suggested that RO5126766 could not promote apoptosis of the HEL^{RE} cells. FIG. 5c reflected that in the HEL^{RE} cells, the drug concentrations (average apoptosis rates) of the groups treated with Agerafenib were: 0 μM (7.60±1.74) (not shown), 0.1 μM (7.05±0.920), 0.3 μM (5.33±0.430), 1 μM (5.11±0.210), 3 μM (7.37±0.220), and 10 μM (8.22±0.390). This suggested that Agerafenib could not promote apoptosis of the HEL^{RE} cells. FIG. 5d reflected that in the HEL^{RE} cells, the drug concentrations (average apoptosis rates) of the groups treated with RAF265 were: 0 μM (6.23±0.300) (not shown), 0.1 μM (6.48±0.480), 0.3 μM (7.31±0.140), 1 μM (8.24±0.730), 3 μM (11.9±0.520), and 10 μM (19.1±3.75). This suggested that RAF265 could promote apoptosis of the HEL^{RE} cells, and this effect increased with increase in the drug concentration. FIG. 5e reflected that in the HEL^{RE} cells, the drug concentrations (average apoptosis rates) of the groups treated with Belvarafenib were: 0 μM (5.15±0.730) (not shown), 0.1 μM (5.55±0.440), 0.3 μM (5.40±0.570), 1 μM (5.71±0.260), 3 μM (6.05±0.430), and 10 μM (7.13±0.450). This suggested that Belvarafenib could not promote apoptosis of the HEL^{RE} cells. The comparison in apoptosis rates of the groups treated with various concentrations of compounds and the group treated with corresponding concentrations of Ruxolitinib was conducted by employing a t-test (*p<0.05, **p<0.01, ***p<0.001).

The above is a detailed introduction to the use of the plurality of compounds provided by the present disclosure in the preparation of a drug for treating myeloproliferative neoplasms. This article uses specific examples to illustrate the principles and implementations of the present disclosure. The description of the aforementioned examples is only used for helping to understand the method of the present disclosure and its core concept. It should be noted that, several improvements and modifications may be made by those of skills in the art without departing from the principle of the present disclosure, and these improvements and modifications should also be considered within the claimed scope of the present disclosure.

1. Use of a compound in preparation of a drug for treating myeloproliferative neoplasms, wherein the compound comprises but is not limited to: one or more of RO5126766, Agerafenib or RAF265.

2. The use according to claim 1, wherein the myeloproliferative neoplasms comprise polycythemia vera, essential thrombocythemia or myelofibrosis, wherein the myelofibrosis comprises primary myelofibrosis, myelofibrosis secondary to polycythemia vera or myelofibrosis secondary to essential thrombocythemia.

3. Use of a compound in preparation of a drug for treating drug-resistant myeloproliferative neoplasms, wherein the compound comprises but is not limited to: one or more of MLN2480, RO5126766, Agerafenib, RAF265 or Belvarafenib.

4. The use according to claim 3, wherein the drug-resistant myeloproliferative neoplasms comprise Ruxolitinib-resistant myeloproliferative neoplasms.

5. The use according to claim 3, wherein the drug-resistant myeloproliferative neoplasms comprise drug-resistant polycythemia vera, drug-resistant essential thrombocythemia or drug-resistant myelofibrosis, wherein the myelofibrosis comprises primary myelofibrosis, myelofibrosis secondary to polycythemia vera or myelofibrosis secondary to essential thrombocythemia.

6. (canceled)

7. (canceled)

8. (canceled)

9. (canceled)

10. The use according to claim 3, wherein the drug further comprises a pharmaceutically-acceptable auxiliary material.

11. (canceled)

12. A method for treating myeloproliferative neoplasms by administering any one or more of the following:
(I). a compound;
(II). the compound and a pharmaceutically-acceptable auxiliary material; or
(III). a drug combination consisting of the compound and any other active ingredient;
wherein the compound comprises but is not limited to: one or more of RO5126766, Agerafenib or RAF265.

13. The method according to claim 12, wherein the myeloproliferative neoplasms comprise polycythemia vera, essential thrombocythemia or myelofibrosis, wherein the myelofibrosis comprises primary myelofibrosis, myelofibrosis secondary to polycythemia vera or myelofibrosis secondary to essential thrombocythemia.

14. A method for treating drug-resistant myeloproliferative neoplasms by administering any one or more of the following:
(I). a compound;
(II). the compound and a pharmaceutically-acceptable auxiliary material; or
(III). a drug combination consisting of the compound and any other active ingredient;
wherein the compound comprises but is not limited to: one or more of MLN2480, RO5126766, Agerafenib, RAF265 or Belvarafenib.

15. The method according to claim 14, wherein the drug-resistant myeloproliferative neoplasms comprise Ruxolitinib-resistant myeloproliferative neoplasms.

16. The method according to claim 14, wherein the drug-resistant myeloproliferative neoplasms comprise drug-resistant polycythemia vera, drug-resistant essential thrombocythemia or drug-resistant myelofibrosis, wherein the myelofibrosis comprises primary myelofibrosis, myelofibrosis secondary to polycythemia vera or myelofibrosis secondary to essential thrombocythemia.

17. (canceled)

18. (canceled)

19. (canceled)

20. (canceled)
